(19) United States
(12) Patent Application Publication
(10) Pub. No.: US 2025/0099627 A1
(43) Pub. Date: Mar. 27, 2025

(54) COMPOSITIONS WITH METAL NANOPARTICLES, THEIR METHODS OF MANUFACTURE AND THEIR USES

(71) Applicant: YELLOWBIRD DIAGNOSTICS INC., Orleans (CA)

(72) Inventors: Adam Jason Shuhendler, Ottawa (CA); Nicholas David Calvert, Ottawa (CA)

(21) Appl. No.: 18/730,403

(22) PCT Filed: Jan. 18, 2023
(86) PCT No.: PCT/CA2023/050051

§ 371 Date: Jul. 19, 2024

Related U.S. Application Data
(60) Provisional application No. 63/300,732, filed on Jan. 19, 2022.

(51) Int. Cl. A61K 49/00; A61K 47/10; A61K 47/18; A61K 47/32; B82Y 5/00; B82Y 15/00

(52) U.S. Cl. CPC A61K 49/0065; A61K 47/10; A61K 47/18; A61K 47/32; A61K 49/0093; A61K 2121/00; B82Y 5/00; B82Y 15/00

(57) ABSTRACT

A composition comprising a plurality of particle clusters in a carrier, at least one particle cluster comprising a plurality of metal nanoparticles, wherein a configuration of the at least one particle cluster is such that the composition has an absorbance spectra peak of above about 900 nm. A method of making the composition comprising: reacting a metal nanoparticle precursor with a stabilizing agent to produce functionalized metal nanoparticles, dispersing the functionalized metal particles in a clustering agent to form the particle clusters; and re-suspending the particle clusters in a carrier to form the composition. Uses of the composition include as a contrast agent for imaging.

### CROSS-REFERENCE TO RELATED APPLICATIONS

This application claims the benefit of priority from U.S. Provisional Application No. 63/300,732, filed Jan. 19, 2022, the contents of which are hereby incorporated by reference in their entirety.

### FIELD

The present disclosure relates to compositions with metal nanoparticles, their methods of manufacture and their uses.

### BACKGROUND

Nanoparticles are used in many applications, including for example, catalysis, energy materials/photonics, and imaging as a contrast agent. Variations in the base material of the nanoparticles, reaction conditions, and surface chemistries can alter their physical, chemical, and optical properties, making them an extremely versatile contrast agent with an extremely wide variety of applications.

One such use is as contrast agents in biomedical imaging using the second near-infrared (NIR-II) window, around 1000 to 1700 nm, where photon penetration in vivo is maximal due to minimized absorption and scattering by blood and tissue. This can allow the capturing of deep-tissue, high signal-to-noise ratio images.

With respect to the medical field, spherical gold nanoparticles (AuNPs) are used for a variety of different imaging modalities, such as OCT, due to their well-established biocompatibility, physicochemical and plasmonic tunability, and targetability. One of the major drawbacks of conventional AuNPs, whether spherical or another shape, is that their localized surface plasmon resonance wavelength (500 nm-650 nm) lies within the visible light spectrum, meaning that it is incompatible with more commonly used imaging modalities that take advantage of the biological imaging window in the NIR-II window (>1000 nm).

More recently, gold-based compositions with resonances above 1000 nm have been proposed for biomedical applications but suffer from a number of disadvantages such as lack of ease of preparation, scalability, and signal strength.

There are other metallic plasmonic materials that can reach these NIR-II wavelengths, such as copper sulfide and silver, but gold remains the standard due to being chemically inert and with well-reported biological clearance. There is a need therefore for improved compositions including nanoparticles.

### SUMMARY

It is an object of the present invention to ameliorate at least some of the deficiencies present in the prior art. Embodiments of the present technology have been developed based on the inventors' appreciation that there is a need for improved compositions that include nanoparticles for imaging purposes.

Inventors have discovered that the optical absorption properties of a composition which includes metal nanoparticles can be tailored by providing clusters of the metal nanoparticles and adapting the properties of the clusters to adapt the optical properties of the composition. Inventors have noted that adapting cluster properties such as one or more of: number of metal nanoparticles in each cluster, a diameter of each cluster, a shape of each cluster, a packing of the metal nanoparticles within each cluster, and a size distribution of the clusters in the carrier can adapt the optical absorption of the composition. In certain embodiments, the composition may have optical properties which are shifted to the second near-infrared (NIR-II) window.

Inventors have hypothesized, without being held to any theory, that such clusters of the metal nanoparticles can undergo a large scale plasmon hybridization. Plasmon hybridization is a phenomenon that occurs when two or more plasmonic fields are brought into close proximity, creating a red-shift in the overall plasmonic absorbance band of the material due to destructive interference.

Furthermore, inventors have developed compositions including such clusters which are scalable, water soluble, and homogeneously distributed and so would be suitable for biomedical applications.

This differs from prior art reports of nanoparticle clusters which were composed of less than ten particles in close proximity, and with a plasmonic red-shift of less than 100 nm. (Liu X, et al. ChemPhysChem. 2007; 8(6):906-912; Nguyen V P, et al. Nat Commun. 2021; 12(1):34; Norman T J, et al. J Phys Chem B. 2002; 106(28):7005-7012; Kim J, et al. Nano Converg. 2020; 7(1):5).

Turek et al. (Turek V A, et al. ACS Photonics. 2016; 3(1):35-42) used a microemulsion technique to form gold superclusters, though the clustering only occurred as a shell to the emulsion core, and resulted in optical properties similar to that of standard gold nanoparticles.

Kwon et al. (Kwon N, et al. Nano Lett. 2018; 18(9):5927-5932) also showed a solvophobic formation of gold superclusters using oleylamine-capped gold nanoparticles made in the flask in a hot oil bath that were aggregated when added to ethanol, but disperse in hexanes or other organic solvents. While these clusters exhibited absorbance in the NIR, they were extremely heterogeneous in terms of size and shape distribution, were not water dispersible, and had no clear, distinct NIR absorbance peak (likely due to heterogeneity of sizes and shapes in the solution).

Furthermore, the inventors have also developed methods for tailoring the size and the shape of the nanoparticle clusters using stabilizing agents and pre-capping solvents. Certain embodiments of such methods exploit a solvophobic effect as a driving force. Embodiments of such methods include “locking” the particle cluster morphology to ensure a homogeneous suspension of the particle cluster in a carrier and the ability to transfer the particle clusters into aqueous solvents without loss of morphology.

From one aspect, there is provided a composition comprising a plurality of particle clusters in a carrier, the at least one particle cluster comprising a plurality of metal nanoparticles, wherein a configuration of the at least one particle cluster is such that the composition has an absorbance spectra peak of above about 900 nm.

In certain embodiments, by metal nanoparticles is meant a particle having a metallic nature such as but not limited to comprising a metal, a metal alloy or a metal oxide. By nanoparticles is meant particles having a size range of about 1 nm to about 500 nm. In some embodiments, nanoparticles have a size range of about 1 nm to about 100 nm.

In certain embodiments, the metal nanoparticles comprise silver or gold particles.

In certain embodiments, at least one of the particle clusters comprises a coating layer. The coating layer comprises a polymer which may be amphiphilic.

In certain embodiments, the plurality of particle clusters has a substantially homogenous size distribution. By substantially homogenous is meant, in certain embodiments, that the composition has a polydispersity index of 0.3 or below, as measured by transmission electron microscopy.

In certain embodiments, the at least one particle cluster is water dispersible.

In certain embodiments, the carrier is an aqueous solution. The carrier may be saline, water or dextrose 5% in water. Such compositions may be used for biomedical applications.

In certain other embodiments, the carrier is a polar organic solvent. Such compositions may be used for non-biomedical applications.

In certain embodiments, each metal nanoparticle in the at least one particle cluster is functionalized with a stabilizing agent. The stabilizing agent may comprise one or more of: an amine, a thiol, or a carboxylic acid head group and hydrophobic tail of any length and degree of saturation. Optionally, the stabilizing agent is oleylamine, octadecenethiol, oleic acid, or a combination thereof.

In certain embodiments, the configuration of the at least one particle cluster comprises one or more of: a given number of the metal nanoparticles in the at least one particle cluster, a size of the at least one particle cluster, a shape of the at least one particle cluster, and a given packing of the metal particles in the at least one particle cluster. In certain embodiments, the at least one particle cluster comprises at least 3 layers of nanoparticles in x y and z planes.

In certain embodiments, the absorbance spectra of the composition is between about 900 nm to about 1700 nm, between about 900 nm and about 1600 nm, between about 900 nm and about 1500 nm, between about 900 nm and about 1400 nm, between about 900 nm and about 1300 nm, 950 nm to about 1700 nm, between about 950 nm and about 1600 nm, between about 950 nm and about 1500 nm, between about 950 nm and about 1400 nm, or between about 950 nm and about 1300 nm, between about 1000 nm to about 1700 nm, between about 1000 nm and about 1600 nm, between about 1000 nm and about 1500 nm, between about 1000 nm and about 1400 nm, between about 1000 nm and about 1300 nm.

In certain embodiments, the at least one particle cluster is substantially spherical.

In certain embodiments, the polymer comprises one or more of a polyethylene glycol, a polyvinylchloride, a poly-l-lysine, a poly lactic acid, a poly(lactic-co-glycolic acid), a polystyrene, and a polyvinylpyrrolidone, and/or block copolymers derived therefrom. In certain embodiments, the block copolymer is derived from polyethylene glycol. In certain embodiments, the block copolymer includes a polyoxyalkylene with saturated or unsaturated alkyl chains (e.g. BRIJ™ families); polyoxyethylene derivatives of saturated or unsaturated fatty acids and/or polyoxyalkylene ether of high molecular weight having water soluble, surface active, and wetting properties (e.g. MYRJ™ families).

In certain embodiments, the coating layer comprises a plurality of coating layers over the at least one particle cluster. In certain embodiments in which the coating layer comprises an amphiphilic polymer, there are provided a plurality of amphiphilic coating layers.

In certain embodiments, the composition further comprises a targeting agent attached to the surface of the particle cluster. In certain embodiments in which there is a coating on the particle cluster, the targeting agent is attached to the coating (e.g. the amphiphilic polymer).

In certain embodiments, the targeting agent comprises one or more of: small molecule ligands, peptides, polymers, nucleic acid construct (including DNA and RNA aptamers), protein, nanobody, affibody, minibody, diabody or antibodies.

In certain embodiments, the targeting agent binds a marker of intravascular inflammation.

In certain embodiments, the targeting agent binds specifically to one or more of P-selectin, E-selectin, and VE-cadherin. In some such embodiments, the targeting agent is a ligand of P-selectin, E-selection or VE-cadherin.

In certain embodiments, the targeting agent comprises a mixture of polymers, the mixture comprising mixing ratios of fucose:sulfate (e.g., 1:2), galactose:sulfate (e.g., 1:2), or fucose:galactose:sulfate (e.g., 1:1:1).

In certain embodiments, an average diameter of the particle cluster is about 250 nm to about 1500 nm, or about 300 nm to about 500 nm, or about 419 nm.

In certain embodiments, the metal nanoparticles are substantially spherical.

In certain embodiments, an average particle size of the metal nanoparticles is in a range of from about 2 nm to about 50 nm. In certain embodiments, the metal nanoparticles have an average particle size of about 9 nm.

From another aspect, there is provided a composition comprising a plurality of particle clusters in a carrier, at least one particle cluster of the plurality of particle clusters comprising a plurality of metal nanoparticles, each metal nanoparticle being functionalized with a stabilizing agent, and wherein the at least one particle cluster has a coating layer. In certain embodiments, the coating layer is a polymer, such as an amphiphilic polymer.

In certain embodiments, a configuration of the metal particles in the at least one particle cluster is such that the composition has a absorbance spectra peak of above about 900 nm.

In certain embodiments, the plurality of particle clusters has a substantially homogenous size distribution. By substantially homogenous is meant, in certain embodiments, that the composition has a polydispersity index of 0.3 or below, as measured by transmission electron microscopy.

In certain embodiments, the at least one particle cluster is water dispersible. In certain embodiments, the carrier is an aqueous solution. In certain embodiments, the carrier is saline, water or dextrose 5% in water.

In other embodiments, the carrier is a polar organic solvent.

In certain embodiments, the stabilizing agent comprises one or more of: an amine, a thiol, a carboxylic acid head group and hydrophobic tail.

In certain embodiments, wherein the configuration of the nanoparticles in the particle cluster comprises a given number of the metal particles in the at least one particle cluster and/or a given packing of the metal particles in the at least one particle cluster. In certain embodiments, at least one particle cluster comprises at least 3 layers of nanoparticles in x y and z planes.

In certain embodiments, the absorbance spectra of the composition is between about 900 nm to about 1700 nm, between about 900 nm and about 1600 nm, between about 900 nm and about 1500 nm, between about 900 nm and about 1400 nm, between about 900 nm and about 1300 nm, 950 nm to about 1700 nm, between about 950 nm and about 1600 nm, between about 950 nm and about 1500 nm, between about 950 nm and about 1400 nm, or between about 950 nm and about 1300 nm, between about 1000 nm to about 1700 nm, between about 1000 nm and about 1600 nm, between about 1000 nm and about 1500 nm, between about 1000 nm and about 1400 nm, between about 1000 nm and about 1300 nm.

In certain embodiments, the at least one particle cluster is substantially spherical.

In certain embodiments, the amphiphilic polymer comprises one or more of a polyethylene glycol, a polyvinylchloride, a poly-l-lysine, a poly lactic acid, a PLGA, a polystyrene, a polyvinylpyrrolidone, and/or block copolymers derived therefrom. In certain embodiments, the block copolymer is derived from polyethylene glycol, such as but not limited to polyoxyalkylene with saturated or unsaturated alkyl chains (e.g. BRIJ™ families); polyoxyethylene derivatives of saturated or unsaturated fatty acids and/or polyoxyalkylene ether of high molecular weight having water soluble, surface active, and wetting properties (e.g. MYRJ™ families).

In certain embodiments, the metal nanoparticles comprise particles which are generally metallic and may comprise for example a metal, a metal alloy or a metal oxide. In certain embodiments, the metal nanoparticles comprise silver or gold particles.

In certain embodiments there are provided a plurality of coating layers over the particle cluster.

In certain embodiments, the composition further comprises a targeting agent attached to the surface of the particle cluster or to the coating (e.g. the amphiphilic polymer).

In certain embodiments, the targeting agent comprises one or more of: small molecule ligands, peptides, polymers, nucleic acid construct (including DNA and RNA aptamers), protein, nanobody, affibody, minibody, diabody or antibodies.

In certain embodiments, the targeting agent binds a marker of intravascular inflammation.

In certain embodiments, the targeting agent binds specifically to one or more of P-selectin, E-selectin, and VE-cadherin. In some such embodiments, the targeting agent is a ligand of P-selectin, E-selection or VE-cadherin.

In certain embodiments, the targeting agent comprises a mixture of polymers, the mixture comprising mixing ratios of fucose:sulfate (e.g., 1:2), galactose:sulfate (e.g., 1:2), or fucose:galactose:sulfate (e.g., 1:1:1).

In certain embodiments, an average diameter of the particle cluster is about 250 nm to about 1500 nm, or about 300 nm to about 500 nm.

In certain embodiments, the metal nanoparticles are substantially spherical.

In certain embodiments, an average particle size of the metal nanoparticles ranges from about 2 to about 50 nm. In certain embodiments, the metal nanoparticles have an average particle size of about 9 nm.

In certain embodiments, the composition is suitable for use as a contrast agent.

From a yet further aspect, there is provided a method of making embodiments of the composition as claimed and described herein. The method comprises: (i) reacting a metal nanoparticle precursor with a stabilizing agent to produce functionalized metal nanoparticles, (ii) dispersing the functionalized metal particles in a clustering agent to form the metal particle clusters; and (iii) re-suspending the metal particle clusters in a carrier to form the composition.

In certain embodiments, the metal nanoparticle is generally metallic and may comprise a metal, a metal alloy or a metal oxide.

The metal may comprise silver or gold, and the metal nanoparticle precursor may comprise a gold particle precursor or a silver particle precursor, respectively. In certain embodiments, the metal particle precursor is HAuCl₄ or AgNO₃.

In certain embodiments, the stabilizing agent comprises one or more of: an amine, a thiol, or a carboxylic acid head group and hydrophobic tail of any length and degree of saturation, and optionally the stabilizing agent is oleylamine, octadecenethiol, oleic acid, or a combination thereof.

In certain embodiments, the clustering agent is an organic solvent. The clustering agent may be one or more of: butanol, ethanol, petroleum ether, butanol-hexanes. In certain embodiments, the clustering agent may include a modified polymer or block copolymer comprising hydrophobic and hydrophilic domains, such as but not limited to Pluronic™ family members such as F127, MYRJ™ and/or BRIJ™ family members such as polyethyleneoxide (40) stearate, and polyvinylpyrrolidone.

In certain embodiments, the reaction comprises heating the metal nanoparticle precursor with the stabilizing agent. The heating may comprise microwave heating. In certain other embodiments, the heating comprises one or more of oven heating, oil bath heating, water bath heating or mantle heating. In certain embodiments, microwave heating can significantly reduce reaction time of cluster formation.

In certain embodiments, the carrier in the composition is an aqueous solution, the method further comprising separating the particle clusters from the clustering agent and suspending them in the aqueous solution.

In certain embodiments, the separating is by centrifugation or by sedimentation. In other embodiments, the separating is by size exclusion chromatography or magnetic separation.

In certain embodiments, the method further comprises coating the metal particle cluster in a coating layer.

From another aspect, there is provided a method of biomedical imaging, comprising administering a contrast agent to a subject and imaging the contrast agent in the subject, wherein the contrast agent comprises the composition as described and/or claimed herein. In certain embodiments, the biomedical imaging comprises optical coherence tomography (OCT). The OCT may comprise intravascular OCT.

In certain embodiments, the imaging relies on NIR light at a wavelength from about 1000 nm to about 1700 nm.

In certain other embodiments, the imaging relies on one or more of: (i) absorption of xrays; (ii) diffraction of xrays; (iii) absorption of light; and (iv) detection by ultrasound transducer.

From a yet further aspect, there is provided the composition as described and/or claimed herein for use in imaging, such as biomedical imaging.

From a yet other aspect, there is provided the composition as described and/or claimed herein for use as a contrast agent. The contrast agent may be used during imaging using modalities such as OCT, x-ray, CT, synchrotron, and photoacoustics.

From a further aspect, there is provided a contrast agent for biomedical imaging comprising the composition as described and/or claimed herein.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIG. 1 is a flow diagram of a method of making a composition with metal nanoparticles, according to certain embodiments of the present technology;

FIG. 2 is a schematic of a method of making a composition with metal nanoparticles, according to certain embodiments of the present technology;

FIG. 3 shows a morphology and size distribution of particle clusters of metal nanoparticles in a composition, according to certain embodiments of the present technology;

FIG. 4 shows an aqueous size and dispersibility of particle clusters of metal nanoparticles in a composition, according to certain embodiments of the present technology;

FIG. 5 shows absorbance spectra of particle clusters of metal nanoparticles, with and without a coating, in a composition, according to certain embodiments of the present technology;

FIG. 6 shows finite difference time domain simulation data of particle clusters of metal nanoparticles in a composition, according to certain embodiments of the present technology;

FIG. 7 shows finite difference time domain simulation data of particle clusters of metal nanoparticles in a composition using fixed unit cell, according to certain embodiments of the present technology;

FIG. 8 shows finite difference time domain simulation data of particle clusters of metal nanoparticles in a composition using a varying unit cell, according to certain embodiments of the present technology;

FIG. 9 shows finite difference time domain simulation data of particle clusters of metal nanoparticles in a composition using a varying coating layer thickness, according to certain embodiments of the present technology;

FIG. 10 shows transmission electron microscopy images and absorbance spectra of a composition comprising metal nanoparticle clusters in saline, according to certain embodiments of the present technology;

FIG. 11 shows intravascular optical coherence tomography images of a composition comprising metal nanoparticle clusters in a carrier compared with reference gold nanoparticles, according to certain embodiments of the present technology;

FIG. 12 shows intravascular optical coherence tomography contrast enhancement between a composition comprising metal nanoparticle clusters in a carrier compared with reference gold nanoparticles, according to certain embodiments of the present technology;

FIG. 13 shows intravascular optical coherence tomography pull back images in a vascular phantom between a composition comprising metal nanoparticle clusters in a carrier compared with reference gold nanoparticles, according to certain embodiments of the present technology;

FIG. 14 shows intravascular optical coherence tomography of Sprague-Dawley rat abdominal aorta sequentially flushed with saline, a composition comprising metal nanoparticle clusters in a carrier, and again with saline, according to certain embodiments of the present technology;

FIG. 15A is a diagram of an approach to functionalize the surface polymers of gold particle clusters (AuSCs) with targeting ligands (1-3), according to certain embodiments of the present technology;

FIG. 15B shows transmission electron micrographs of AuSCs functionalized with different combination of targeting ligands as indicated (left panel), according to certain embodiments of the present technology. The degree of AuSC binding to P-selectin in vitro for different formulations of AuSC targeting is shown in the right panel. *p<0.05, **p<0.01, ****p<0.001;

FIG. 15C shows intravascular optical coherence tomography of Sprague-Dawley rat abdominal aorta after induction of intra-arterial inflammation before and after the introduction of untargeted (left) AuSC, or targeted AuSC (middle and right). Arteries were sequentially flushed with saline, a composition comprising metal nanoparticle clusters in a carrier, and again with saline, according to certain embodiments of the present technology;

FIG. 15D shows 400 MHz 1H NMR spectrum of as-synthesized FucoPEO prior to particle conjugation, according to certain embodiments of the present technology;

FIG. 15E shows 400 MHz 1H NMR spectrum of as-synthesized GalaPEO prior to particle conjugation, according to certain embodiments of the present technology;

FIG. 15F shows 400 MHz 1H NMR spectrum of as-synthesized SulfoPEO prior to particle conjugation, according to certain embodiments of the present technology;

FIG. 15G shows MALDI-TOF spectra for FucoPEO. The spectra shows the central mass of M+Na, with other peaks being different ethylene oxide chain lengths with the same functionalization, according to certain embodiments of the present technology;

FIG. 15H shows MALDI-TOF spectra for GalaPEO. The spectra shows the central mass of M+Na, with other peaks being different ethylene oxide chain lengths with the same functionalization, according to certain embodiments of the present technology; and

FIG. 15I shows MALDI-TOF spectra for SulfoPEO. The spectra shows the central mass of M+Na, with other peaks being different ethylene oxide chain lengths with the same functionalization, according to certain embodiments of the present technology.

### DETAILED DESCRIPTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The terms “derivative” and “variant” are used interchangeably herein.

Aspects of the present technology comprise compositions having clusters of metal nanoparticles in a carrier. Optical properties, such as absorption spectra, of the composition can be tailored to a given use by adapting one or more cluster parameters, for example, cluster diameter, number of nanoparticles in the cluster, packing of the metal nanoparticles in the cluster and a size or shape distribution of the clusters in the carrier. Aspects of the present technology comprise methods of making such compositions.

### Compositions

In certain embodiments, the composition comprises particle clusters comprising metal nanoparticles in a carrier.

### Metal Nanoparticles and Particle Clusters

The metal nanoparticles may comprise any suitable metallic particle, such as for example metal alloys, metal oxides and pure metals. Example metals include, but are not limited to: gold, silver, copper, palladium, manganese oxide. A precursor to the gold particles may comprise HAuCl₄.

In certain embodiments, the metal particles have a diameter within a range of about 1 to 100 nm, about 1 to about 90 nm, about 1 to about 80 nm, about 1 nm to about 70 nm, about 1 nm to about 60 nm, about 1 nm to about 50 nm, or about 2 nm to about 50 nm. In certain embodiments, the metal nanoparticles comprise gold nanoparticles with a diameter of about 5-15 nm, or about 9 nm.

A size distribution of the metal nanoparticles within a cluster may be substantially homogenous. For example, the metal nanoparticle diameter may range between about 8 nm and about 11 nm, with a median and a mean diameter of 9 nm. In other embodiments, a size distribution of the metal nanoparticles within a cluster may be substantially heterogenous. For example, the metal nanoparticles may have a diameter between about 1 nm to about 100 nm.

The particle clusters within the composition are substantially spherical in certain embodiments.

In certain embodiments, the size of at least some of the particle clusters ranges from about 250 nm to about 1500 nm, about 300 nm to about 1400 nm, about 300 nm to about 1300 nm, about 300 nm to about 1200 nm, about 300 nm to about 1100 nm about 300 nm to about 1000 nm, about 300 nm to about 900 nm, about 300 nm to about 800 nm, about 400 nm to about 800 nm, about 500 nm to about 800 nm, about 300 nm to about 700 nm, about 300 nm to about 600 nm, about 300 nm to about 500 nm, about 400 nm to about 600 nm, or about 400 nm to about 500 nm.

A size of the particle clusters may be measured by any known method such as image analysis of electron microscopy images of the particle clusters or using a particle sizer of the particle clusters in solution.

The particle clusters are substantially homogeneously sized in certain embodiments. By substantially homogenous is meant, in certain embodiments, that the composition has a polydispersity index of 0.3 or below, as measured by transmission electron microscopy.

A packing of the metal nanoparticles may be defined as an interparticle distance. In certain embodiments, the interparticle distance is defined as a unit cell volume with a distance between corners of the unit cell representing the interparticle distance between two metal nanoparticles.

For particle clusters comprising gold nanoparticles, it can be assumed that the unit cell is a face centered cubic unit cell (i.e. unit cell lengths in the x, y and z directions are equal). The unit cell volume, and hence the packing of the gold nanoparticles in the particle cluster, may be determined from a measured size of the particle clusters and the gold nanoparticles. An edge length of the unit cell may be calculated as 2.828*atomic radius of gold (144) which amounts to 0.4073 nm.

In certain embodiments, the packing of the metal nanoparticles is homogenous through the supercluster. In other embodiments, the packing of the metal particles varies from an interior portion to an exterior portion of the particle cluster. For example, the metal nanoparticles may be more closely packed in the interior portion of the particle cluster compared to the exterior portion of the particle cluster.

In certain embodiments, the packing of the metal nanoparticles a unit cell volume of one or more of 13 nm³, 14 nm³ or 15 nm³. For example, the packing in the interior portion of the particle cluster may comprise a unit cell volume of 14 nm³ and the packing in the exterior portion of the particle cluster may comprise 15 nm³. In another example, the packing of the metal particles extending radially from the interior portion to the exterior portion may be graduated, such as having a unit cell volume of 13 nm³, 14 nm³ and 15 nm³.

The absorbance spectra of the composition is between about 900 nm to about 1700 nm, between about 900 nm and about 1600 nm, between about 900 nm and about 1500 nm, between about 900 nm and about 1400 nm, between about 900 nm and about 1300 nm, 950 nm to about 1700 nm, between about 950 nm and about 1600 nm, between about 950 nm and about 1500 nm, between about 950 nm and about 1400 nm, or between about 950 nm and about 1300 nm, between about 1000 nm to about 1700 nm, between about 1000 nm and about 1600 nm, between about 1000 nm and about 1500 nm, between about 1000 nm and about 1400 nm, between about 1000 nm and about 1300 nm. In certain embodiments, the absorbance spectra of the composition is above about 900 nm.

An absorption spectra of the composition has a peak wavelength between about 800 nm to 1400 nm, about 800 nm, about 900 nm, about 1000 nm, about 1100 nm, about 1200 nm, about 1300 nm or about 1400 nm.

Absorbance spectra, such as plasmonic resonance spectra, can be obtained using any suitable instrument and method, such as but not limited to Visible Near Infrared spectrometer. The spectra of the carrier may be subtracted from the spectra of the entire composition.

In certain embodiments, at least some of the clusters of metal nanoparticles have a coating layer encapsulating the cluster. There may be provided a plurality of coating layers, such as 2, 3 or 4 coating layers. The coating layer may comprise a polymer, a block copolymer or a modified polymer. The coating layer may comprise an amphiphilic polymer. In certain embodiments, the coating layer is polyoxyethylene (40) stearate (e.g. “Myrj 52”).

In certain embodiments, the coating layer has a thickness if about 0.5 nm to about 10 nm. In certain embodiments, the thickness of the coating layer is less than about 1 nm.

The coating layer may comprise any suitable polymer such as one or more of a polyethylene glycol, a polyvinylchloride, a poly-l-lysine, a poly lactic acid, a poly(lactic-co-glycolic acid), a polystyrene, and a polyvinylpyrrolidone. The coating layer may comprise a block copolymer such as any member of the MYRJ™ and BRIJ™ families, such as but not limited to polyoxyalkylene with saturated or unsaturated alkyl chains (e.g. BRIJ™ families); polyoxyethylene derivatives of saturated or unsaturated fatty acids and/or polyoxyalkylene ether of high molecular weight having water soluble, surface active, and wetting properties (e.g. MYRJ™ families).

In certain embodiments, at least some of the clusters comprise metal nanoparticles functionalized with a stabilizing agent. In certain embodiments, the stabilizing agent comprises one or more of: an amine, a thiol, or a carboxylic acid head group and hydrophobic tail of any length and degree of saturation, and optionally the stabilizing agent is oleylamine, octadecenethiol, oleic acid, or a combination thereof. In certain embodiments, certain of the clusters of the composition comprise 9 nm gold particles capped with oleylamine.

In certain embodiments, there is provided a targeting agent attached to the surface of the particle cluster. In certain embodiments in which there is a coating on the particle cluster, the targeting agent is attached to the coating (e.g. the amphiphilic polymer). The targeting agent may comprise one or more of: small molecule ligands, peptides, polymers, nucleic acid construct (including DNA and RNA aptamers), protein, nanobody, affibody, minibody, diabody or antibodies.

The targeting agent may bind a marker of intravascular inflammation. The targeting agent may bind to one or more of P-selectin, E-selectin, and VE-cadherin. The targeting agent may be a ligand of P-selectin, E-selectin or VE-cadherin, such as without limitation a mixture of polymers, for example and without limitation, comprising mixing ratios of fucose:sulfate (1:2), galactose:sulfate (1:2) or fucose:galactose:sulfate (1:1:1).

### Carrier

The carrier may comprise any suitable carrier. In certain embodiments, the carrier comprises an aqueous solution, cream or gel. For biomedical uses, the aqueous carrier may comprise one or more of saline, water or dextrose solution.

In certain other embodiments, the carrier comprises an organic solvent.

### Methods

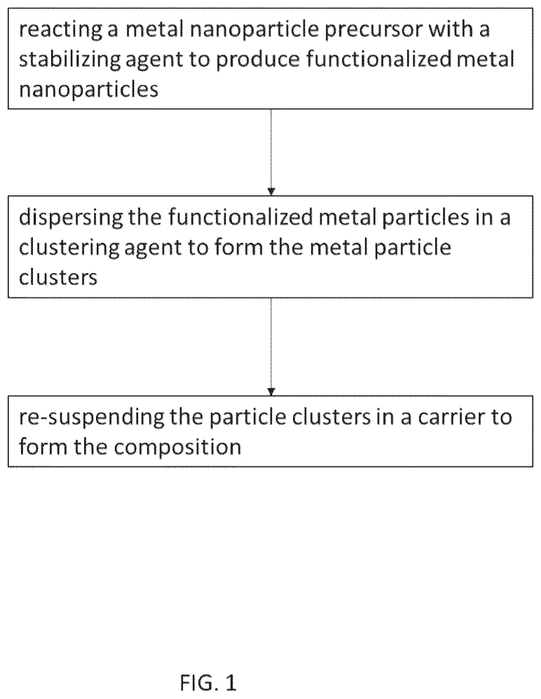

Referring to FIG. 1, in certain aspects, the method for making embodiments of the composition comprise: (i) reacting a metal nanoparticle precursor with a stabilizing agent to produce functionalized metal nanoparticles, (ii) dispersing the functionalized metal particles in a clustering agent to form the metal particle clusters; and (iii) re-suspending the metal particle clusters in a carrier to form the composition.

Any suitable metal nanoparticle precursor, stabilizing agent, clustering agent and carrier can be used to generate metal nanoparticle clusters with different physical, optical, and chemical properties.

In certain embodiments, metal nanoparticle precursors may comprise HAuCl₄ or AgNO₃. However, other metal salts are also possible as metal precursors.

In certain embodiments, the stabilizing agent comprises one or more of an amine, a thiol, or a carboxylic acid head group and hydrophobic tail of any length and degree of saturation. Optionally the stabilizing agent is oleylamine, octadecenethiol, oleic acid, or a combination thereof.

In certain embodiments, the clustering agent is an organic solvent. Examples of clustering agent include, but are not limited to, butanol, ethanol, petroleum ether, butanol-hexanes with or without pluronic F127, polyethyleneoxide (40) stearate, and polyvinylpyrrolidone.

In certain embodiments, the reaction comprises heating the metal nanoparticle precursor with the stabilizing agent. The heating can be performed in any manner and to any suitable temperature for any suitable length time sufficient to permit functionalization of the metal particles with the stabilizing agent. The manner of heating is not particularly limited. For example, the heating can be one or more of microwave heating, oven heating, oil bath heating, water bath heating or mantle heating.

The method further comprises, in certain embodiments, coating the metal particle cluster with a coating layer. The coating layer may comprise a polymer, such as an amphiphilic polymer. The polymer may comprise one or more of a polyethylene glycol, a polyvinylchloride, a poly-l-lysine, a poly lactic acid, a poly(lactic-co-glycolic acid), a polystyrene, and a polyvinylpyrrolidone. The polymer may comprise a modified polymer and/or a block copolymer thereof. The block copolymer may comprise hydrophobic and hydrophilic domains (i.e. amphipathic), such as but not limited to pluronic family members such as F127, MYRJ™ and/or BRIJ™ family members such as polyethyleneoxide (40) stearate, and polyvinylpyrrolidone. The coating step may be repeated to coat the particle cluster in a plurality of coatings.

In certain embodiments, the coating layer comprises a plurality of coating layers over the at least one particle cluster. In certain embodiments in which the coating layer comprises an amphiphilic polymer, there are provided a plurality of amphiphilic coating layers. It was found that addition of the polymer layer may increase a packing of the particle cluster.

In certain embodiments in which the carrier in the composition is an aqueous carrier, the method further comprises separating the particle clusters from the clustering agent and suspending them in the aqueous carrier. The separating may be by one or more of centrifugation, sedimentation, size exclusion chromatography or magnetic separation.

In certain embodiments, the method further comprises attaching a targeting agent to the surface of the particle cluster or to the coating layer (when it is present). The targeting agent may be any suitable agent such as, but not limited to, small molecule ligands, peptides, polymers, nucleic acid construct (including DNA and RNA aptamers), protein, nanobody, affibody, minibody, diabody or antibodies. The targeting agent may bind a marker of intravascular inflammation such as P-selectin, E-selectin, or VE-cadherin. For example and without limitation, the targeting agent may be a ligand of P-selectin, E-selectin or VE-cadherin, e.g., a mixture of polymers, e.g. a mixture of polymers comprising mixing ratios of fucose:sulfate (e.g., 1:2), galactose:sulfate (e.g., 1:2), or fucose:galactose:sulfate (e.g., 1:1:1).

In certain embodiments, the method comprises forming a particle cluster having a given size by selecting an appropriate hydrophilicity of the clustering agent. More specifically, the size of the particle cluster can be increased by selecting a clustering agent with higher hydrophilicity.

Uses of compositions of the present technology are not limited and may include as contrast agents for imaging, and the like.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1—Gold-Based Particle Clusters

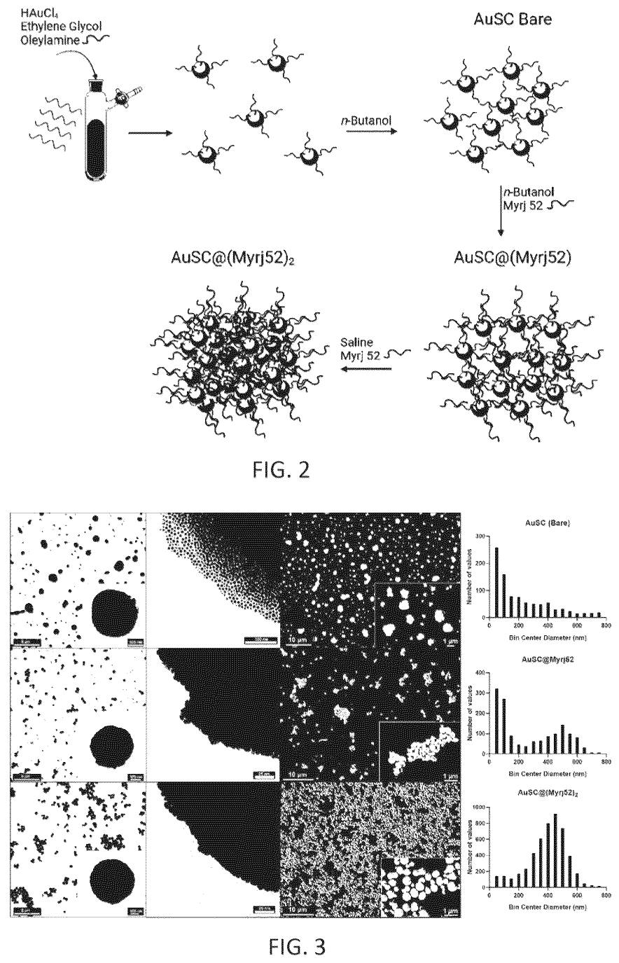

Referring to FIG. 2 a 40 mM solution of HAuCl₄ in ethylene glycol (4 mL) (metal particle precursor) was added to 24.3 mM of oleylamine (8 mL) (stabilizing agent) and 8 mL of ethylene glycol under stirring (950 RPM) in a three-neck flask. Two of the three necks were capped with septa, while the center neck was connected to a vacuum distillation adapter connected to a vacuum and an empty 5 mL flask. The three-neck flask was heated to 43° C. while a vacuum was applied, and the solution was stirred for ˜30 min until all effervescence had ceased. The flask was flushed with nitrogen gas and the vacuum adaptor was removed. The solution was carefully poured (10 mL) into two microwave reaction vessels which were flushed with nitrogen. The vessels underwent microwave synthesis in a chemical microwave (CEM Discover) (75 W power heating to 115° C., then holding this temperature for 90 s and cooling back to 50° C. on release). The vessels were decanted into 50 mL Falcon tubes containing a 15 g/L of polyethyleneoxide stearate (Myrj 52) solution in n-butanol (coating layer). This solution was shaken overnight, centrifuged three times (1000 xg, 10 min, RT), resuspended by sonication into butanol (clustering agent) for the first two centrifugations, then finally resuspended in a 40 g/L polyethyleneoxide stearate (Myrj 52) solution (coating layer) in ultrapure water by sonication. This solution was again shaken overnight, centrifuged three times under the same conditions and resuspended into fresh ultrapure water after each centrifugation. This final solution was passed through a size exclusion chromatography column (SEC) to remove excess polymer. This final composition comprising particle clusters in suspension was stored at 4° C.

FIG. 3 shows morphology and size distribution of the particle clusters throughout their synthesis in butanol clustering with no polymer coating (A), a single polymer coating suspended in butanol (B), and twice-polymer coated in water (C). Size distributions were acquired through automated particle size analysis with ImageJ from two synthetic replicates for clusters, each with three regions on the transmission electron microscopy grid counted.

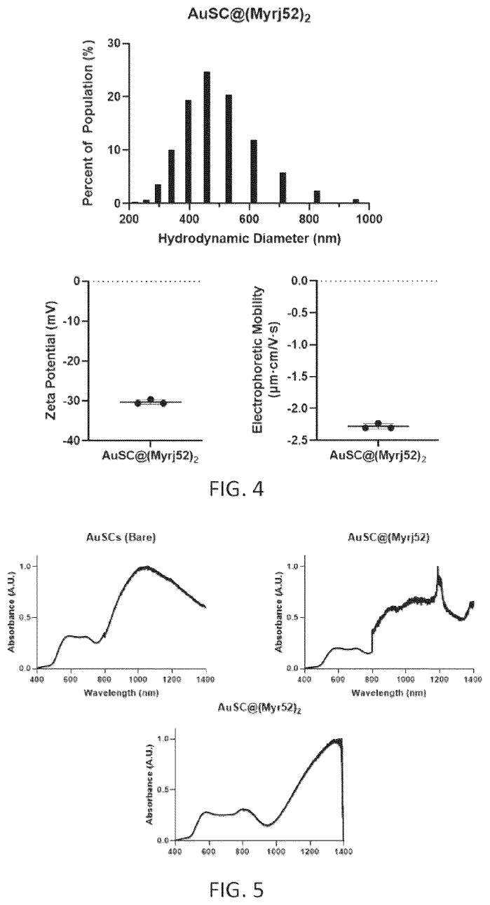

FIG. 4 shows aqueous size and dispersibility of the particle clusters made of gold nanoparticles and double coated with an amphiphilic polymer coating layer (AuSC@(Myrj 52)₂). (A) distribution of hydrodynamic sizes of AuSC@(Myrj 52)₂. (B) zeta potential of AuSC@(Myrj 52)₂. (C) electrophoretic mobility of AuSC@(Myrj 52)₂. All data was acquired from triplicate readings of an AuSC@(Myrj 52)₂ solution in distilled water.

### Example 2—Tailoring Optical Properties of the Composition

Optical properties of the particle clusters of Example 1 dispersed in butanol without a polymer coating (AuSC bare) and with one or two polymer coatings of polyethylene oxide (40) stearate (AuSC@(Myrj52), AuSC@Myrj52)₂ respectively) were compared (FIG. 5). The particle clusters without a polymer coating showed a plasmon peak similar to the distinct peak known for single gold nanoparticles around 9 nm (˜550 nm), but with a bathochromic shift spanning 550 to 700 nm (FIG. 5A). The more notable optical feature is the broad absorbance peak spanning from 800 nm to 1400 nm. Both of these peaks persisted in butanol after the single polymer coating of the particle clusters (FIGS. 5A and B, respectively). The NIR peak became much sharper and more refined after further size focusing and aqueous transfer in the AuSC@(Myrj 52)₂ spectra when dispersed in water, though the large NIR broadband peak was retained from 1000 to 1400 nm (FIG. 5C).

The formation of the large NIR peak was thought to be the result of large scale plasmon hybridization throughout the 450 nm diameter particle clusters, resulting in a red-shifted plasmonic band. The hybridization of such a large number of gold nanoparticles in one structure being held in close proximity was the reason for the much larger change in absorbance band compared to the plasmonic hybridization red-shifts reported in prior literature. The unique optical properties of the double-coated gold particle clusters (AuSC@(Myrj 52)₂) are thought to derive from the large number of individual gold nanoparticles that assemble, which are entirely composed of tightly packed gold nanoparticles from core to surface. This composition was confirmed through FIB-SEM images of AuSC@(Myrj 52) 2 clusters where superclusters were etched to reveal their core architecture.

This example demonstrates how the optical properties of the composition can be tailored by coating, or not, the particle clusters.

### Example 3. In Silico Simulation

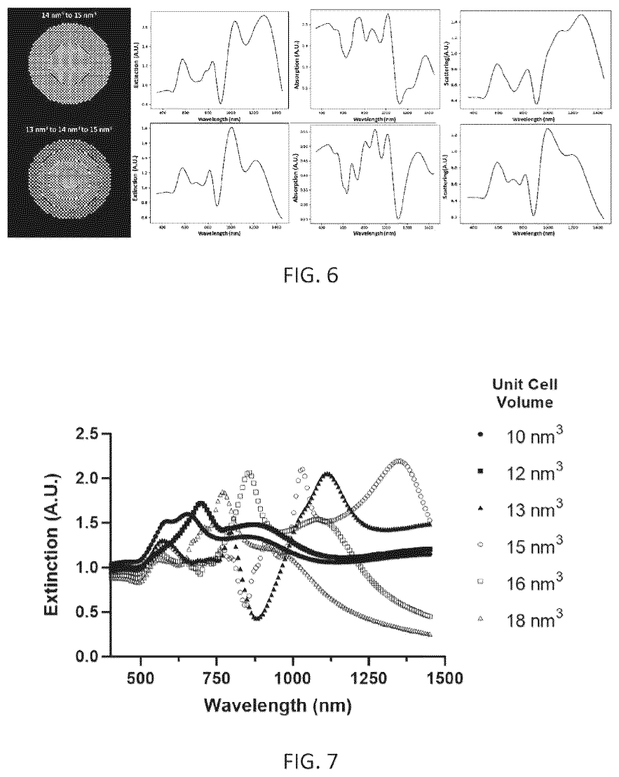

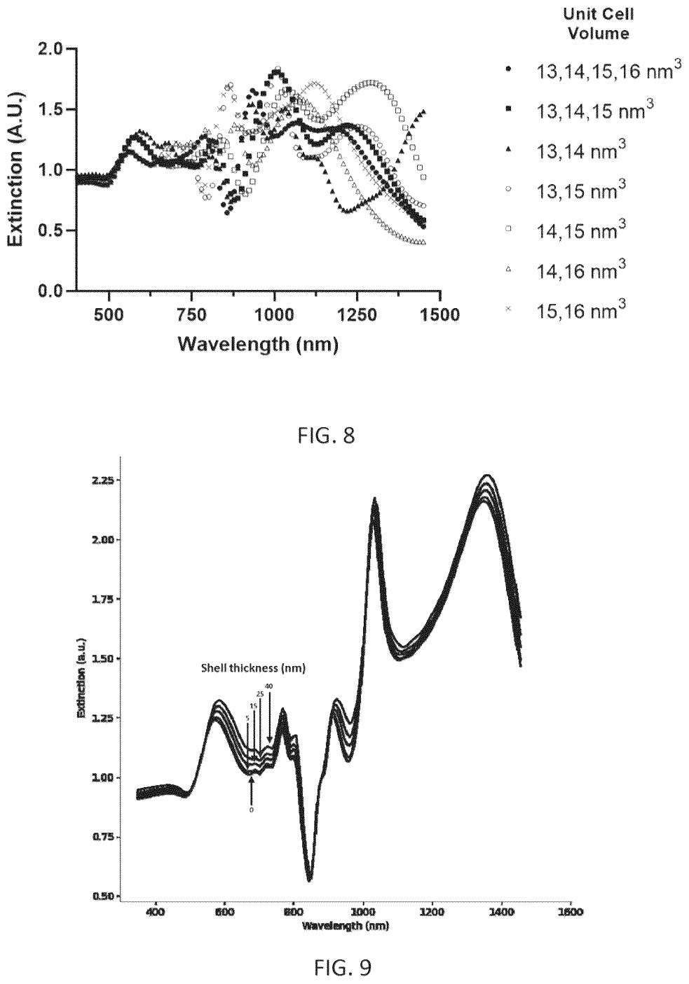

In order to better observe how large-scale hybridization was the result of the large NIR peak in the double coated particle clusters of Example 2 (AuSC@(Myrj 52)₂), these clusters were simulated in silico using finite difference time domain (FDTD) models with the aim to reproduce the particle clusters with similar optical properties to that observed experimentally. These simulations were also used to determine the volume of the unit cell that the particle clusters created, since evaluation by X-ray diffraction did not produce signals other than the unit cells for standard gold nanoparticles. It is thought that similar to the molecular grain of a gold nanoparticle, the particle clusters likely assemble themselves in a unit-cell-like fashion. Since the unit cell volume ultimately dictates the distance between particles, it is a measure which can help understand how gold nanoparticle packing can affect optical properties of the particle cluster. The simulated particle clusters were based on gold nanoparticles which were oleylamine capped and driven to cluster using an amphiphilic solvent. The simulated particle clusters were assumed to be polymer coated with polyethyleneoxide (40) stearate in butanol, and then again in water, which was likely to result in a higher degree of polymer coating on the constituent gold nanoparticles nearer to the solvent-exposed surface, and a higher amount of oleylamine on particles closer to the cluster core. The surface heterogeneity of constituent gold nanoparticles would result in a heterogeneous set of unit cells throughout the supercluster, with core gold nanoparticles having a smaller unit cell than those at the surface. When this gradient unit cell cluster was simulated in FDTD, with either two or three different unit cells, it produced absorbance spectra nearly identical to what was observed experimentally. Two different simulations using an interior unit cell volume of 14 nm³ and exterior of 15 nm³ (FIG. 6A) or from interior to exterior of 13 nm³, 14 nm³, and 15 nm³ (FIG. 6B) resulted in extremely similar absorbance spectra, especially compared to the experimental data. Clusters of a homogeneous unit cell volume were dissimilar to the experimental data (FIG. 7). Both absorption and scattering components of photophysical behaviour of the simulated particle clusters were extracted from extinction data, demonstrating that while scattering is the dominant interaction from the visible to NIR wavelengths, there is still a large degree of light absorption in the visible range. Importantly, scattering is the dominant mode of light interaction in the NIR-II. It is this dual mode of light interaction (i.e. both absorption and scattering) across the visible-NIR-NIR-II spectrum that makes these clusters an interesting material with a broad range of uses. FIG. 8 shows finite difference time domain simulation data of AuSC@(Myrj 52)₂ superclusters using a varying unit cell. Each curve represents the unit cell volume range (from interior to exterior) of a simulated particle cluster and the resulting extinction (absorption and scattering combined) spectra. Simulations are in an environment of water with a 10 nm polymer (Myrj 52) coating around the particle cluster.

FIG. 9 shows finite difference time domain simulation data of particle clusters with a double polymer layer (AuSC@(Myrj 52)₂) using a varying polymer coating thickness. Clusters were simulated by finite difference time domain measurements with a 15 nm³ unit cell composed of 9 nm diameter gold nanoparticles. The surrounding simulation environment was water. The thickness of the polymer coating, simulated up to 50 nm thick, did not alter the optical properties of the particle clusters.

### Example 4—Tailoring Optical Properties of the Composition Using Different Stabilizing Agent and Different Clustering Agent

Table 1 shows different synthetic conditions for formation of gold particle clusters including reaction concentrations and conditions, workup steps, and the resulting absorbance peak and appearance of superclusters. As can be seen, the optical properties of the composition can be tailored by adapting the reagents used to make the composition.

TABLE 1

| Gold | | | Reaction | Reaction | | | | |
|---|---|---|---|---|---|---|---|---|
| Precursor | | | Hold | Hold | Post- | | | |
| Concn. | Stabilizing | Microwave | Time | Temp. | Microwave | Transfer | Transfer | |
| (mM) | Agent | Power(W) | (s) | (° C.) | Diluent | Method | Solvent | Purification |
| 20 | Oleylamine | 300 | 1 | 70 | Ethanol | Centrifugation | Ethanol | N/A |
| | (3M) | | | | | (11,300 xg, 1 hr) | | |
| 20 | Oleylamine | 300 | 1 | 100 | Ethanol | Centrifugation | Ethanol | N/A |
| | (3M) | | | | | (11,300 xg, 1 hr) | | |
| 20 | Oleylamine | 300 | 1 | 120 | Ethanol | Centrifugation | Ethanol | N/A |
| | (3M) | | | | | (11,300 xg, 1 hr) | | |
| 20 | Oleylamine | 300 | 1 | 150 | Ethanol | Centrifugation | Ethanol | N/A |
| | (3M) | | | | | (11,300 xg, 1 hr) | | |
| 20 | Oleylamine | 300 | 1 | 170 | Ethanol | Centrifugation | Ethanol | N/A |
| | (3M) | | | | | (11,300 xg, 1 hr) | | |
| 20 | Oleylamine | 300 | 1 | 190 | Ethanol | Centrifugation | Ethanol | N/A |
| | (3M) | | | | | (11,300 xg, 1 hr) | | |
| 20 | Oleylamine | 300 | 300 | 150 | Ethanol | Centrifugation | Ethanol | N/A |
| | (3M) | | | | | (125 xg, 1 hr) | | |
| 20 | Oleylamine | 300 | 300 | 225 | Ethanol | Centrifugation | Ethanol | N/A |
| | (3M) | | | | | (125 xg, 1 hr) | | |
| 20 | Oleylamine | 300 | 900 | 150 | Ethanol | Centrifugation | Ethanol | N/A |
| | (3M) | | | | | (125 xg, 1 hr) | | |
| 20 | Oleylamine | 300 | 900 | 225 | Ethanol | Centrifugation | Ethanol | N/A |
| | (3M) | | | | | (125 xg, 1 hr) | | |
| 20 | Oleylamine | 300 | 1800 | 150 | Ethanol | Centrifugation | Ethanol | N/A |
| | (3M) | | | | | (125 xg, 1 hr) | | |
| 20 | Oleylamine | 300 | 1800 | 225 | Ethanol | Centrifugation | Ethanol | N/A |
| | (3M) | | | | | (125 xg, 1 hr) | | |
| 20 + | Oleylamine | 300 | 900 | 200 | Ethanol | Centrifugation | Ethanol | N/A |
| Entry 12 | (3M) | | | | | (125 xg, 1 hr) | | |
| AuSCs | | | | | | | | |
| 20 | Octadecenethiol | 300 | 900 | 225 | Ethanol | Centrifugation | Ethanol | N/A |
| | (3M) | | | | | (125 xg, 1 hr) | | |
| 20 | Oleylamine | 300 | 1 | 100 | Ethanol | Sedimentation | Ethanol | N/A |
| | (3M) | | | | | by gravity | | |
| 20 | Oleylamine | 300 | 900 | 100 | Ethanol | Sedimentation | Ethanol | N/A |
| | (3M) | | | | | by gravity | | |
| 20 | Oleylamine: | 300 | 900 | 150 | Ethanol | Sedimentation | Ethanol | N/A |
| | Octadecenethiol | | | | | by gravity | | |
| | (3 M, 1:1) | | | | | | | |
| 20 | Oleylamine | 100 | 60 | 115 | Ethanol | Centrifugation | Ethanol | N/A |
| | (3M) | | | | | (1000 xg, 10 | | |
| | | | | | | min) | | |
| 20 | Oleylamine | 100 | 150 | 115 | Ethanol | Centrifugation | Ethanol | N/A |
| | (3M) | | | | | (1000 xg, 10 | | |
| | | | | | | min) | | |
| 20 | Oleylamine | 100 | 300 | 115 | Ethanol | Centrifugation | Ethanol | N/A |
| | (3M) | | | | | (1000 xg, 10 | | |
| | | | | | | min) | | |
| 20 | Oleylamine | 100 | 150 | 115 | Petroleum | Centrifugation | Petroleum | N/A |
| | (3M) | | | | Ether | (1000 xg, 10 | ether | |
| | | | | | | min) | | |
| 20 | Oleylamine | 75 | 300 | 115 | 12.5 g/L | Centrifugation | Ethanol | Centrifugation |
| | (1.5M) | | | | Polyethylene- | (1000 xg, 10 | | (1000 xg, 10 |
| | | | | | oxide (40) | min) | | min) |
| | | | | | stearate in | | | |
| | | | | | butanol | | | |
| 20 | Oleylamine | 75 | 300 | 115 | 12.5 g/L 35 | Centrifugation | Ethanol | Centrifugation |
| | (1.5 M) | | | | kDa | (1000 xg, 10 | | (1000 xg, 10 |
| | | | | | polyvinyl- | min) | | min) |
| | | | | | pyrrolidone | | | |
| | | | | | in butanol | | | |
| 20 | Oleylamine | 75 | 300 | 115 | 20:80 | Centrifugation | 20:80 | N/A |
| | (1.5M) | | | | Butanol: | (1000 xg, 10 | Butanol: | |
| | | | | | Hexanes | min) | Hexanes | |
| 20 | Oleylamine | 75 | 300 | 115 | 50:50 | Centrifugation | 50:50 | N/A |
| | (1.5 M) | | | | Butanol: | (1000 xg, 10 | Butanol: | |
| | | | | | Hexanes | min) | Hexanes | |
| 20 | Oleylamine | 75 | 300 | 115 | Butanol | Centrifugation | Butanol | N/A |
| | (1.5M) | | | | | (1000 xg, 10 | | |
| | | | | | | min) | | |
| 20 | Oleylamine | 75 | 300 | 115 | 12.5 g/L 35 | Centrifugation | Ethanol | Centrifugation |
| | (0.75M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 20 | Oleylamine | 75 | 300 | 115 | 12.5 g/L 35 | Centrifugation | Ethanol | Centrifugation |
| | (3M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 20 | Oleylamine | 75 | 150 | 115 | 12.5 g/L35 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 10 | Oleylamine | 75 | 150 | 115 | 12.5 g/L35 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 40 | Oleylamine | 75 | 150 | 115 | 12.5 g/L 35 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 40 | Oleylamine | 75 | 150 | 115 | 15 g/L 10 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 40 | Oleylamine | 75 | 150 | 115 | 15 g/L 55 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrrolidone in | | | |
| | | | | | butanol | | | |
| 40 | Oleylamine | 75 | 150 | 115 | 15 g/L 360 | Centrifugation | Butanol | Centrifugation |
| | (1.5M) | | | | kDa | (1000 xg, 10 | | (1000 xg, 10 |
| | | | | | polyvinyl- | min) | | min) |
| | | | | | pyrolidone in | | | |
| | | | | | butanol | | | |
| 40 | Oleylamine | 75 | 150 | 115 | 15 g/L | Centrifugation | Butanol | Centrifugation |
| | (1.5M) | | | | 1300 kDa | (1000 xg, 10 | | (1000 xg, 10 |
| | | | | | polyvinyl- | min) | | min) |
| | | | | | pyrolidone in | | | |
| | | | | | butanol | | | |
| 40 | Oleylamine | 75 | 150 | 115 | 15 g/L | Centrifugation | Butanol | Centrifugation |
| | (1.5M) | | | | Polyethylene- | (1000 xg, 10 | | (1000 xg, 10 |
| | | | | | oxide (40) | min) | | min) |
| | | | | | stearate in | | | |
| | | | | | butanol | | | |
| 40 | Oleylamine | 75 | 150 | 115 | 15 g/L | Centrifugation | Butanol | Centrifugation |
| | (1.5M) | | | | Pluronic | (1000 xg, 10 | | (1000 xg, 10 |
| | | | | | F127 in | min) | | min) |
| | | | | | butanol | | | |
| 40 | Oleylamine | 75 | 150 | 115 | 15 g/L 360 | Centrifugation | Butanol + | Centrifugation |
| | (1.5M) | | | | kDa | (1000 xg, 10 | secondary | (1000 xg, 10 |
| | | | | | polyvinyl- | min) | stabilization | min), |
| | | | | | pyrrolidone in | | A | Pd10 column |
| | | | | | butanol | | | purification |
| 40 | Oleylamine | 75 | 150 | 115 | 15 g/L | Centrifugation | Butanol + | Centrifugation |
| | (1.5M) | | | | Polyethylene | (1000 xg, 10 | secondary | (1000 xg, 10 |
| | | | | | oxide (40) | min) | stabilization | min), |
| | | | | | stearate in | | A | Pd10 column |
| | | | | | butanol | | | purification |
| 40 | Oleylamine | 75 | 150 | 115 | 15 g/L 360 | Centrifugation | Butanol + | Centrifugation |
| | (1.5M) | | | | kDa | (1000 xg, 10 | secondary | (1000 xg, 10 |
| | | | | | polyvinyl- | min) | stabilization | min), |
| | | | | | pyrrolidone in | | B | Pd10 column |
| | | | | | butanol | | | purification |
| 40 | Oleylamine | 75 | 150 | 115 | 15 g/L | Centrifugation | Butanol + | Centrifugation |
| | (1.5M) | | | | Polyethylene | (1000 xg, 10 | secondary | (1000 xg, 10 |
| | | | | | oxide (40) | min) | stabilization | min), |
| | | | | | stearate in | | B | Pd10 column |
| | | | | | butanol | | | purification |

| Gold | | Center | | | | |
|---|---|---|---|---|---|---|
| Precursor | | Peak | | | | |
| Concn. | Suspension | SPR | Diameter | Degree of | Cluster | |
| (mM) | Solvent | (nm) | (nm) | Clustering | Circularity | Distribution |
| 20 | Ethanol | Low NIR | 10 | Individual | N/A | Homogeneous |
| | | Activity | | nanoparticles | | |
| | | | | (~10 nm) | | |
| 20 | Ethanol | Low NIR | 500 | Very diffuse | Circular | Heterogeneous |
| | | activity | | | | |
| 20 | Ethanol | 1150 nm | 600 | Very diffuse | Circular | Heterogeneous |
| | | (Broad) | | | | |
| 20 | Ethanol | 1250 nm | 600 | Diffuse | Uneven | Heterogeneous |
| | | (Very | | | circular | |
| | | broad) | | | | |
| 20 | Ethanol | 1250 nm | 600 | Somewhat | Uneven | Heterogeneous |
| | | (Very | | diffuse | circular | |
| | | broad) | | | | |
| 20 | Ethanol | 1250 nm | 800 | Somewhat | Abstract | Heterogeneous |
| | | (Very | | diffuse | | |
| | | broad) | | | | |
| 20 | Ethanol | 1150 | 600 | Somewhat | Uneven | Heterogeneous |
| | | (Broad) | | diffuse | circular | |
| 20 | Ethanol | 1250 | 800 | Somewhat | Abstract | Heterogeneous |
| | | (Broad) | | diffuse | | |
| 20 | Ethanol | 1150 | 600 | Tight | Uneven | Heterogeneous |
| | | (Broad) | | | circular | |
| 20 | Ethanol | 1250 | 800 | Tight | Abstract | Heterogeneous |
| | | (Broad) | | | | |
| 20 | Ethanol | 1150 | 700 | Tight | Intercluster | Heterogeneous |
| | | (Broad) | | | packing | |
| 20 | Ethanol | 1250 | 800 | Tight | Intercluster | Heterogeneous |
| | | (Broad) | | | packing | |
| 20 + | Ethanol | N/A | 1000 | Tight | Uneven | Homogeneous |
| Entry 12 | | | | | circular | |
| AuSCs | | | | | | |
| 20 | Ethanol | N/A | 600 | Very tight | Uneven | Homogeneous |
| | | | | | circular | |
| 20 | Ethanol | N/A | 500 | Somewhat | Uneven | Homogeneous |
| | | | | diffuse | circular | |
| 20 | Ethanol | N/A | >1500 | Tight | Chains of | Heterogeneous |
| | | | | | circular | |
| | | | | | clusters | |
| 20 | Ethanol | 1450 | 350 | Very tight | Urchin-like, | Heterogeneous |
| | | (Sharp) | | | circular | |
| 20 | Ethanol | N/A | 400 | Diffuse | Uneven | Heterogeneous |
| | | | | | circular, | |
| | | | | | chains of | |
| | | | | | clusters | |
| 20 | Ethanol | N/A | 300 | Diffuse | Uneven | Heterogeneous |
| | | | | | circular, | |
| | | | | | chains of | |
| | | | | | clusters | |
| 20 | Ethanol | N/A | 350 | Somewhat | Uneven | Heterogeneous |
| | | | | diffuse | circular, | |
| | | | | | chains of | |
| | | | | | clusters | |
| 20 | Petroleum | N/A | 500 | Very diffuse | Circular, | Homogeneous |
| | ether | | | | disassembling | |
| | | | | | clusters | |
| 20 | Ethanol | 1150 | 400 | Very tight | Circular | Homogeneous |
| | | (Broad) | | | | |
| 20 | Ethanol | 1150 | 450 | Very tight | Circular | Homogeneous |
| | | (Broad) | | | | |
| 20 | 20:80 | 520 | 600 | Very diffuse | Circular, | Heterogeneous |
| | Butanol: | (Sharp) | | | disassembling | |
| | Hexanes | | | | clusters | |
| 20 | 50:50 | N/A | 400 | Diffuse | Uneven | Heterogeneous |
| | Butanol: | | | | circular, | |
| | Hexanes | | | | chains of | |
| | | | | | disassembling | |
| | | | | | clusters | |
| 20 | Butanol | N/A | >1500 | Very tight | Abstract | Heterogeneous |
| | | | | | chains of | |
| | | | | | clusters | |
| 20 | Ethanol | N/A | 250 | Very tight | Abstract | Heterogeneous |
| | | | | | chains of | |
| | | | | | clusters | |
| 20 | Ethanol | N/A | 300 | Very tight | Uneven | Homogeneous |
| | | | | | circular | |
| 20 | Butanol | 1300 | 250 | Very tight | Circular | Homogeneous |
| | | (Broad) | | | | |
| 10 | Butanol | 950 | 400 | Very tight | Circular, | Homogeneous |
| | | (Very | | | jagged | |
| | | broad) | | | edges | |
| 40 | Butanol | 1100 | 500 | Very tight | Circular | Homogeneous |
| | | (Very | | | | |
| | | broad) | | | | |
| 40 | Butanol | 850 | 450 | Very tight | Circular | Homogeneous |
| | | (Broad) | | | | |
| 40 | Butanol | 1100 | 450 | Very tight | Circular | Homogeneous |
| | | (Broad) | | | | |
| 40 | Butanol | 1000 | 450 | Very tight | Circular | Homogeneous |
| | | (Broad) | | | | |
| 40 | Butanol | 1000 | 600 | Diffuse | Circular, | Heterogeneous |
| | | (Broad) | | | disassembling | |
| | | | | | clusters | |
| 40 | Butanol | 1300 | 250 | Tight | Uneven | Heterogeneous |
| | | (Broad) | | | circular, | |
| | | | | | aggregating | |
| | | | | | clusters | |
| 40 | Butanol | 1250 | 450 | Very tight | Circular | Homogeneous |
| | | (Broad) | | | | |
| 40 | Distilled | 1250 | 400 | Very tight | Circular | Homogeneous |
| | water | (Broad) | | | | |
| 40 | Distilled | 1250 | 450 | Very tight | Circular | Homogeneous |
| | water | (Broad) | | | | |
| 40 | Distilled | 1150 | 400 | Tight | Circular, | Heterogeneous |
| | water | (Broad) | | | some | |
| | | | | | budding | |
| 40 | Distilled | 1150 | 450 | Very tight | Circular, | Heterogeneous |
| | water | (Broad) | | | some | |
| | | | | | budding | |

### Example 5—Composition for Use as IV-OCT

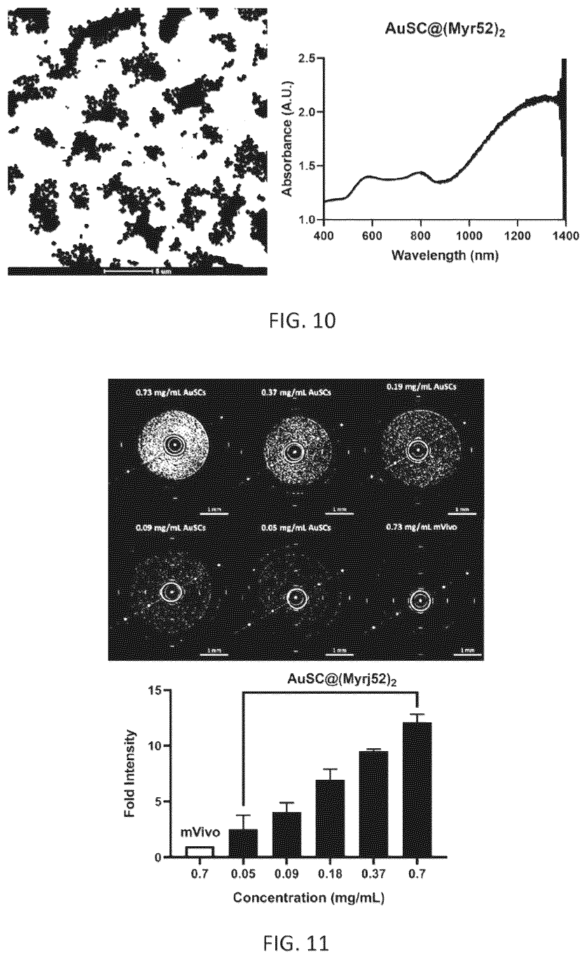

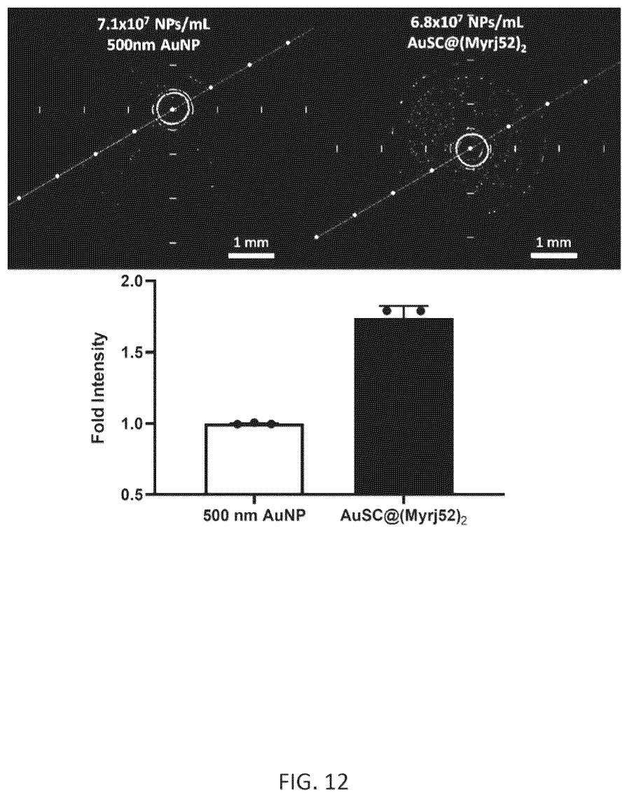

Intravascular optical coherence tomography (IV-OCT) is commonly used in interventional cardiological assessments to image the health of coronary vessels and guide stent placement. However, IV-OCT is limited to anatomical imaging since no contrast agents (agents that can provide specific signal enhancements) currently exist. IV-OCT relies on backscattered incoherent NIR-II light (center wavelength is around 1300 nm), which is well-suited to contrast enhancement by gold particle clusters. The composition of Example 1 which included double-coated particle clusters of cold in a saline carrier (AuSC@(Myrj 52)₂) were prepared. The strong ionic solvent had no effect on the structure or optical properties of the particle clusters (FIG. 10). Two-dimensional (2D) IV-OCT scans on different concentrations of the particle clusters suspended in a glass pipette were performed to evaluate contrast enhancement effects (FIG. 11). A saline soluble, commercially available gold nanoparticle solution (m Vivo, Medilumine Inc.) was used as reference sample. The enhancement in IV-OCT signal generated by the composition was significantly greater (>10-fold) than that generated by the reference gold nanoparticles, even after normalizing to total gold content of the solution (FIG. 11). Even microgram amounts of AuSC@(Myrj 52)₂ produced a three-fold signal enhancement. Large discrete 500 nm gold nanoparticles (not superclusters) were also evaluated and did not show a strong signal compared to the AuSC@(Myrj 52) 2 clusters of the composition (˜1.8-fold signal enhancement with the superclusters compared to AuNPs, normalized to number of particles in solution) (FIG. 12).

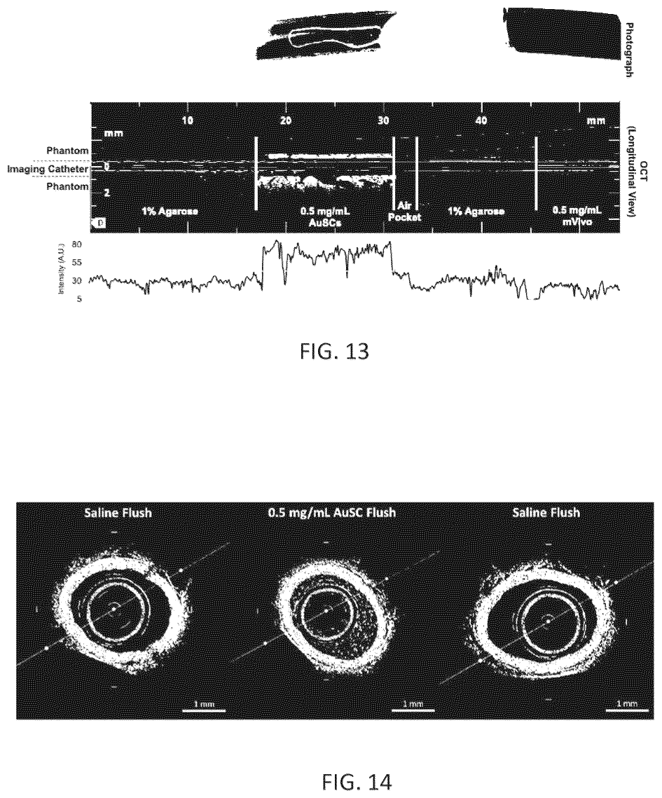

Particle clusters including metal nanoparticles of the present technology and gold nanoparticles in a suspension of agarose were used to prepare a vascular phantom (FIG. 13) to evaluate the more commonly used dynamic implementation of IV-OCT, where an imaging catheter is pulled back through vasculature to generate a longitudinal image (FIG. 13). The intensity of the signal over the distance of the scan was mapped (FIG. 13), with only the particle clusters of the composition producing a contrast enhancement above background. The particle clusters also highlight a significant amount of detail within the agarose, such as air pockets and breaks that aren't readily discernable in the absence of the contrast agent.

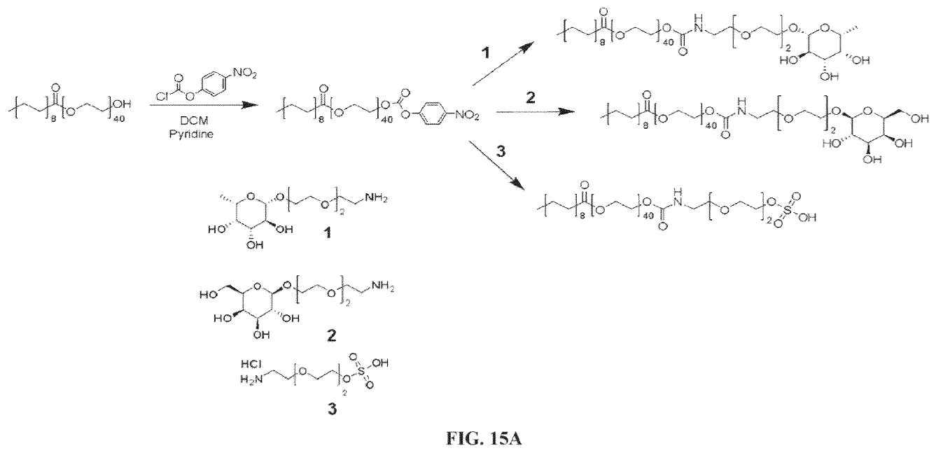

The composition including the AuSC@(Myrj 52)₂ particle clusters were also applied to in vivo imaging of the abdominal aorta (AA) of a Sprague-Dawley rat (FIG. 14). The AA was imaged while being flushed with saline (FIG. 14), then imaged while being flushed with a 0.5 mg/mL AuSC@(Myrj 52)₂ solution in saline (FIG. 14). There is a clear contrast to the flushed space that is differentiable from the signal created from the walls of the AA. We then flushed the AA with more saline to demonstrate the particles could easily be washed out of the field of view after imaging (FIG. 14).

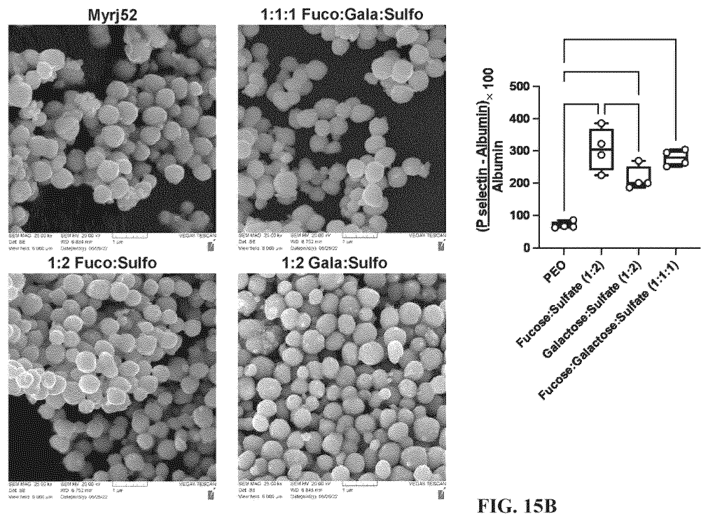

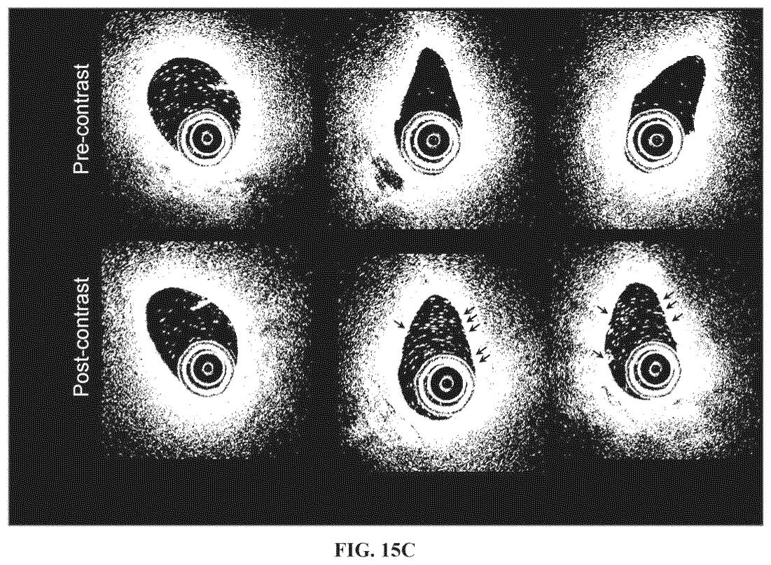

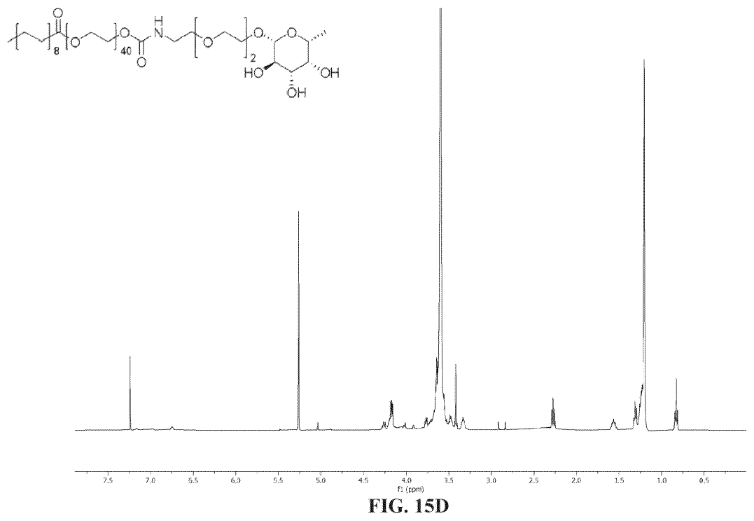

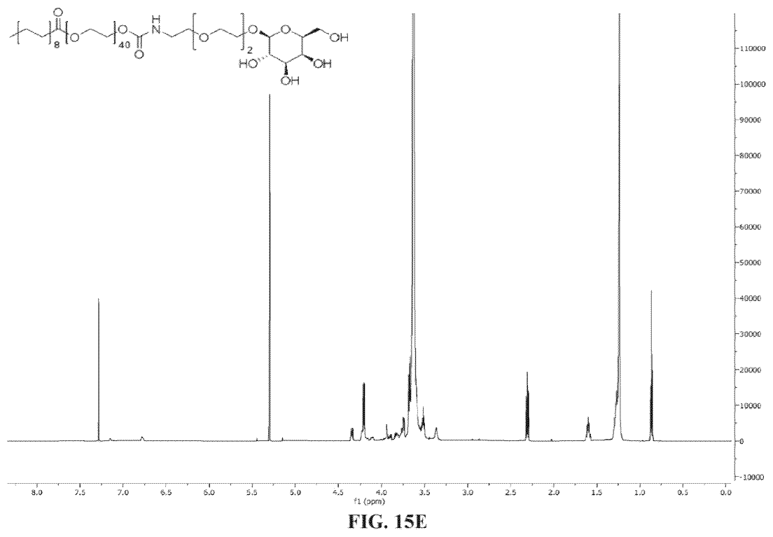

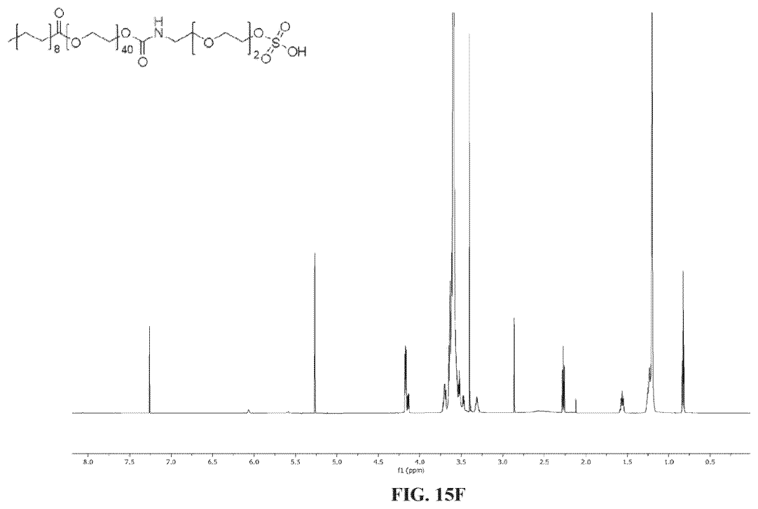

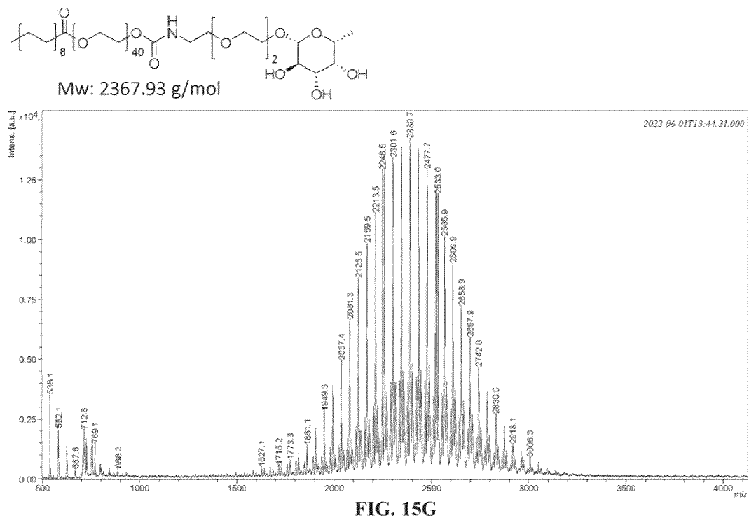

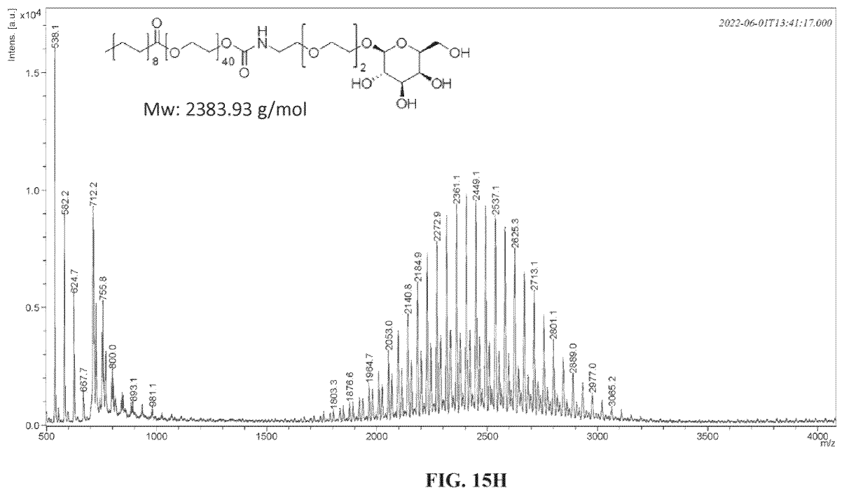

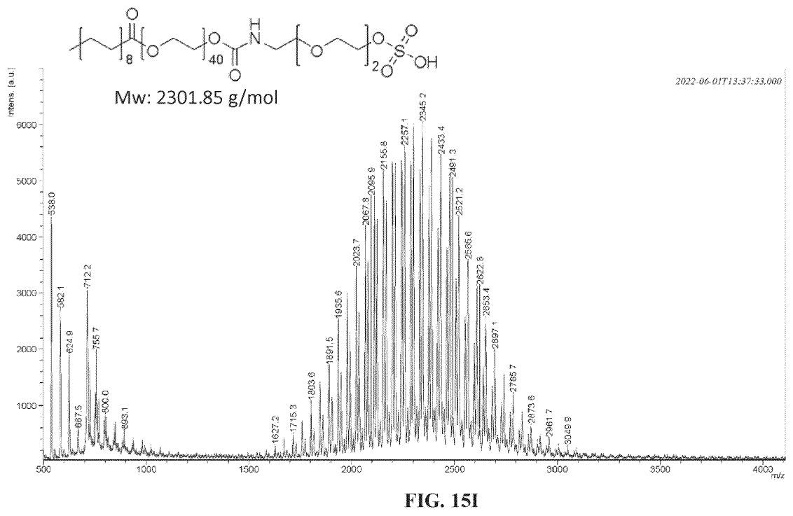

With the addition of a targeting group on the surface of the particle clusters specific for markers of inflammation, this difference in contrast from pre and post flushes could be used to detect intravascular inflammation before major morphological changes occur (FIGS. 15A-I). The polymer coating can readily be functionalized with targeting groups that can bind biomolecular targets of interest (i.e. P-selectin, E-selectin, VE-cadherin, etc.), all markers of intravascular inflammation, affording molecular imaging by IV-OCT (FIG. 15A). Targeting agents could be small molecule ligands, peptides, aptamers, or antibodies conjugated to the polymer coating using well established mechanisms (for example, lbrich K et al. Chem Rev. 2016; 116(9):5338-5431, the contents of which are herein incorporated by reference).

FIG. 15B shows transmission electron micrographs of AuSCs functionalized with different combinations of targeting ligands, as indicated (left), and the degree of AuSC binding to P-selectin in vitro for different formulations of AuSc targeting (right). FIG. 15C shows intravascular optical coherence tomography of Sprague-Dawley rat abdominal aorta after induction of intra-arterial inflammation before and after the introduction of untargeted (left) AuSC, or targeted AuSC (middle and right), demonstrating successful targeting of the AuSCs functionalized with targeting ligands.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

1.-63. (canceled)

64. A composition comprising:
a plurality of particle clusters in a carrier, the particle clusters comprising a plurality of metal nanoparticles functionalized with a stabilizing agent, the particle clusters having a coating layer;
wherein the stabilizing agent comprises one or more of an amine, a thiol, or a carboxylic acid head group, and a hydrophobic tail;
wherein the coating layer comprises an amphiphilic polymer;
wherein the metal nanoparticle comprises a plasmonic metal, a plasmonic metal alloy or a plasmonic metal oxide; and
wherein the carrier comprises an aqueous solution or a polar organic solvent.

65. The composition of claim 64, wherein the metal nanoparticles are silver particles or gold particles having a size range of about 1 nm to about 500 nm, or a size range of about 1 nm to about 100 nm, and wherein an average diameter of the particle cluster is about 250 nm to about 1500 nm, or about 300 nm to about 500 nm.

66. The composition of claim 64, wherein the stabilizing agent is oleylamine, octadecenethiol, oleic acid, or a combination thereof.

67. The composition of claim 64, wherein the amphiphilic polymer comprises one or more of a polyethylene glycol, a polyvinylchloride, a poly-L-lysine, a poly lactic acid, a poly(lactic-co-glycolic acid), a polystyrene, a polyvinylpyrrolidone, or a modified polymer or a block copolymer thereof.

68. The composition of claim 64, wherein the amphiphilic polymer comprises polyoxyalkylene with saturated or unsaturated alkyl chains (e.g. BRIJ™ families); polyoxyethylene derivatives of saturated or unsaturated fatty acids and/or polyoxyalkylene ether of high molecular weight having water soluble, surface active, and wetting properties (e.g. MYRJ™ families).

69. The composition of claim 64, further comprising a targeting agent attached to the surface of the particle cluster or to the coating, wherein the targeting agent comprises one or more of: small molecule ligands, peptides, polymers, nucleic acid construct (including DNA and RNA aptamers), protein, nanobody, affibody, minibody, diabody or antibodies.

70. The composition of claim 69, wherein the targeting agent binds a marker of intravascular inflammation, or wherein the targeting agent binds to one or more of P-selectin, E-selectin, and VE-cadherin.

71. The composition of claim 69, wherein the targeting agent comprises a mixture of polymers, the mixture comprising mixing ratios of fucose:sulfate (1:2), galactose:sulfate (1:2) or fucose:galactose:sulfate (1:1:1).

72. The composition of claim 64, wherein the carrier comprises saline solution, water, or dextrose 5% in water.

73. The composition of claim 64, wherein the metal nanoparticle is a gold nanoparticle, the stabilizing agent is oleylamine, the coating layer comprises Myrj™ 52, and the carrier comprises water.

74. A method of making the composition as defined in claim 64, the method comprising:
(i) providing a solution of a metal nanoparticle precursor with a stabilizing agent;
(ii) reacting the metal nanoparticle precursor with the stabilizing agent to produce functionalized metal nanoparticles;
(iii) suspending the functionalized metal nanoparticles in a solution comprising a coating agent and a clustering agent to provide coated metal particle clusters; and
(iv) suspending the coated metal particle clusters in a carrier to provide the composition;
wherein the stabilizing agent comprises one or more of an amine, a thiol, or a carboxylic acid head group, and a hydrophobic tail;
wherein the coating layer comprises an amphiphilic polymer;
wherein the metal nanoparticle precursor comprises a plasmonic metal precursor, a plasmonic metal alloy precursor or a plasmonic metal oxide precursor;
wherein the carrier comprises an aqueous solution or a polar organic solvent; and
wherein the clustering agent comprises an organic solvent.

75. The method of claim 74, wherein the metal nanoparticle precursor is a gold particle precursor or a silver particle precursor.

76. The method of claim 75, wherein the metal nanoparticle precursor is HAuCl₄ or AgNO₃.

77. The method of claim 74, wherein the amphiphilic polymer comprises one or more of a polyethylene glycol, a polyvinylchloride, a poly-L-lysine, a poly lactic acid, a poly(lactic-co-glycolic acid), a polystyrene, a polyvinylpyrrolidone, or a modified polymer or a block copolymer thereof.

78. The method of claim 74, wherein the step of reacting comprises heating the metal nanoparticle precursor with the stabilizing agent, wherein the heating is one or more of microwave heating, oven heating, oil bath heating, water bath heating or mantle heating.

79. The method of claim 74, wherein the stabilizing agent is oleylamine, octadecenethiol, oleic acid, or any combination thereof.

80. The method of claim 74, wherein the clustering agent is one or more of butanol, ethanol, petroleum ether, and butanol-hexanes.

81. The method of claim 74, wherein the carrier in the composition is an aqueous solution, the method further comprises a step of separating the particle clusters from the clustering agent and suspending the separated particle clusters in the aqueous solution, wherein the step of separating is carried out by one or more of centrifugation, sedimentation, size exclusion chromatography or magnetic separation.

82. The method of claim 74, wherein the metal nanoparticle precursor is a HAuCl₄, the stabilizing agent is oleylamine, the coating agent comprises Myrj™ 52, the clustering agent is n-butanol, and the carrier is water.

83. A method of biomedical imaging, comprising administering a contrast agent to a subject and imaging the contrast agent in the subject, wherein the contrast agent comprises the composition as defined in claim 64, wherein the particle clusters are water dispersible.

84. The method of claim 83, wherein the biomedical imaging comprises optical coherence tomography (OCT), including intravascular OCT.

85. The method of claim 83, wherein said imaging relies on NIR light at a wavelength from about 1000 nm to about 1700 nm.

86. The method of claim 83, wherein said imaging relies on one or more of:
(i) absorption of x-rays;
(ii) diffraction of x-rays;
(iii) absorbance of light; and
(iv) detection by ultrasound transducer.
